(19) United States
(12) Patent Application Publication
(10) Pub. No.: US 2021/0253533 A1
(43) Pub. Date: Aug. 19, 2021

(54) DISUBSTITUTED 5(3)-PYRAZOLE CARBOXYLATES AND A PROCESS FOR THEIR PREPARATION FROM ENOLATES AND FLUOROALKYLAMINOREAGENTS (FAR) REAGENTS

(71) Applicant: Bayer Aktiengesellschaft, Leverkusen (DE)

(72) Inventors: Sergii PAZENOK, Leichlingen (DE); Anton LISHCHYNSKYI, Solingen (DE); Arnd NEEFF, Burscheid (DE); Winfried ETZEL, Leichlingen (DE)

(21) Appl. No.: 17/252,785

(22) PCT Filed: Jun. 14, 2019
(86) PCT No.: PCT/EP2019/065644

§ 371 Date: Dec. 16, 2020

(30) Foreign Application Priority Data
Jun. 18, 2018 (EP) 18178287.1

(51) Int. Cl. C07D 231/18; C07C 315/04

(52) U.S. Cl. CPC C07D 231/18; C07C 317/46; C07C 315/04

(57) ABSTRACT

The present invention relates to disubstituted 5(3)-pyrazole carboxylates of the formula (Ia) or (Ib) and a process for their preparation

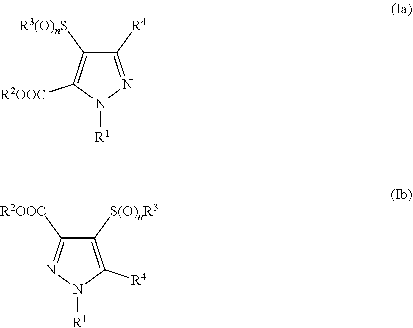

wherein R¹, R², R³, R⁴ and n are defined as above.

The present invention relates to disubstituted 5(3)-pyrazole carboxylates and a novel process for their preparation. It is known from WO 2012/126766 that N-alkyl-3-haloalkyl-4-(methylsulfinyl)-5-pyrazole carboxylates are important precursors for the synthesis of pyrazole carboxyamides which possess strong insecticidal activity. The chemical synthesis of a pyrazole with C₂F₅-group in position 3 and SMe-group in position 4 was described in WO 2012/126766. This synthesis however requires multi step transformations with moderate yield and tedious isolation and purification.

The utilization of Fluoroalkylaminoreagents (FAR) for synthesis of diverse substituted pyrazoles have been already published for example in Pazenok et al., European Journal of Organic Chemistry 2015(27), 6052-6060; Pazenok et al., European Journal of Organic Chemistry 2013(20), 4249-4253; WO 2014/033164 and WO 2008/022777.

In the light of the prior art described above, it is an object of the present invention to provide a process that does not have the aforementioned disadvantages and hence gives a route to disubstituted 5(3)-pyrazole carboxylates derivatives in high yields.

The object described above was achieved by a process for the preparation of disubstituted 5(3)-pyrazole carboxylates of the formula (Ia) or (Ib),

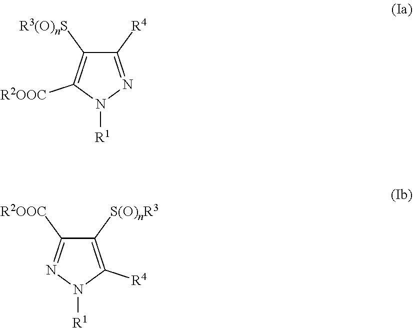

wherein
R¹ is selected from H, (C₁-C₆)alkyl, phenyl or 2-pyridyl,
R² is selected from H, (C₁-C₁₂)alkyl or (C₃-C₈)cycloalkyl,
R³ is selected from (C₁-C₁₂)alkyl, (C₁-C₃)haloalkyl, (C₃-C₈)cycloalkyl, (C₆-C₁₂)aryl, (C₁-C₃)alkyl(C₆-C₁₂)aryl and (C₆-C₁₂)aryl(C₁-C₆)alkyl,
R⁴ is selected from (C₁-C₆)haloalkyl and (C₁-C₃)haloalkoxy(C₁-C₆)haloalkyl and
n is 0, 1 or 2,
comprising a step (A), wherein
α,α-difluoroalkylamino reagents (FAR) of the formula (III),

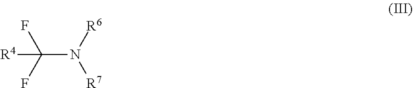

wherein
R⁴ is defined as above and
R⁶ and R⁷ are each independently selected from (C₁-C₆)alkyl, (C₃-C₈)cycloalkyl, (C₆-C₁₂)aryl or together with the nitrogen atom to which they are bonded may form a five- or six-membered ring,

are first converted into compounds of the formula (VI) in the presence of a Lewis Acid [L]

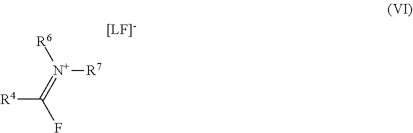

where R⁴, R⁶ and R⁷ are defined as for formula (III) and [LF]⁻ is an anion formed from the Lewis Acid [L] and one fluorine atom from compound (III),

and then the compounds of the formula (VI) are reacted with enolates of the formula (II),

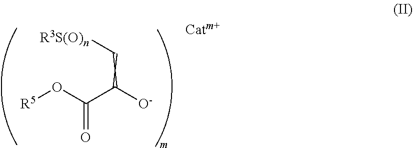

wherein
R⁵ is selected from (C₁-C₁₂)alkyl or (C₃-C₈)cycloalkyl,
R³ and n are defined as above,
m is 1 or 2 and
Cat^{m+} is selected from alkaline metal cations (with m=1), alkaline earth metal cations (with m=2), organic ammonium cations (with m=1) or organic phosphonium cations (with m=1),

to form compounds of formula (IV),

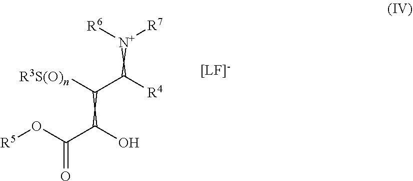

wherein

n, [LF]⁻, R³, R⁴, R⁵, R⁶ and R⁷ are defined as above,

and further comprising a step (B), wherein cyclization with an hydrazine of the formula (V)

NH₂—NH—R¹ (V),

wherein R¹ is defined as above,

takes place to form the compounds of formula (Ia) or (Ib).

Preferred is a process according to the invention, where the radicals in formula (Ia), (Ib), (II), (III), (IV), (V) and (VI) are defined as follows:
R¹ is selected from H, (C₁-C₆)alkyl, phenyl or 2-pyridyl,
R² is selected from H, (C₁-C₆)alkyl or (C₃-C₆)cycloalkyl,
R³ is selected from (C₁-C₆)alkyl, (C₁-C₃)haloalkyl, (C₃-C₆)cycloalkyl, (C₆-C₉)aryl, (C₁-C₃)alkyl(C₆-C₉)aryl and (C₆-C₉)aryl(C₁-C₃)alkyl,
R⁴ is selected from (C₁-C₆)haloalkyl and (C₁-C₃)haloalkoxy(C₁-C₆)haloalkyl,
R⁵ is selected from (C₁-C₆)alkyl or (C₃-C₆)cycloalkyl,
n is 0, 1 or 2,
R⁶ and R⁷ are each independently selected from (C₁-C₆)alkyl, (C₃-C₆)cycloalkyl, (C₆-C₁₂)aryl or
R⁶ and R⁷ together with the nitrogen atom to which they are bonded may form a five- or six-membered ring,
m is 1 and
Cat^{m+} is selected from alkaline metal cations, preferably from Li⁺, Na⁺, K⁺ and Cs⁺, organic ammonium cations, preferably (R⁸)₄N⁺ or organic phosphonium cations, preferably (phenyl)₄P⁺, wherein
R⁸ are each independently selected from (C₁-C₆)alkyl.

More preferred is a process according to the invention, where the radicals in formula (Ia), (Ib), (II), (III), (IV), (V) and (VI) are defined as follows:
R¹ is selected from H, (C₁-C₆)alkyl or phenyl,
R² is selected from H or (C₁-C₆)alkyl,
R³ is selected from (C₁-C₆)alkyl,
R⁴ is selected from (C₁-C₆)haloalkyl and (C₁-C₃)haloalkoxy(C₁-C₆)haloalkyl, wherein the halogen is selected from fluoro and/or chloro,
R⁵ is selected from (C₁-C₆)alkyl,
n is 0, 1 or 2,
R⁶ and R⁷ are each independently selected from (C₁-C₆)alkyl,
m is 1 and
Cat^{m+} is selected from Li⁺, Na⁺, K⁺, Cs⁺ or Me₄N⁺.

Even more preferred is a process according to the invention, where the radicals in formula (Ia), (Ib), (II), (III), (IV), (V) and (VI) are defined as follows:
R¹ is selected from H, methyl, ethyl or phenyl
R² is selected from H, methyl or ethyl,
R³ is selected from methyl or ethyl
R⁴ is selected from difluoromethyl (CF₂H), chlorofluoromethyl (CHFCl), 1,2,2,2-tetrafluoroethyl (CF₃CFH), pentafluoroethyl (C₂F₅) and trifluoromethoxyfluoromethyl (CF₃OCFH),
R⁵ is selected from (C₁-C₃)alkyl,
n is 2,
R⁶ and R⁷ are methyl,
m is 1 and
Cat+ is selected from Li⁺, Na⁺ or K⁺.

Most preferred is a process according to the invention, where the radicals in formula (Ia), (Ib), (II), (III), (IV), (V) and (VI) are defined as follows:
R¹ is selected from H, methyl or phenyl,
R² is selected from H, methyl or ethyl,
R³ is methyl,
R⁴ is selected from difluoromethyl (CF₂H), chlorofluoromethyl (CHFCl), 1,2,2,2-tetrafluoroethyl (CF₃CFH), pentafluoroethyl (C₂F₅) and trifluoromethoxyfluoromethyl (CF₃OCFH),
R⁵ is selected from methyl or ethyl,
n is 2,
R⁶ and R⁷ are methyl,
m is 1 and
Cat^{m+} is Na⁺ or K⁺.

In a particularly preferred embodiment of the present invention n is 2 for the compounds of the general formula (Ia), (Ib), (II) and (IV).

In a preferred embodiment of the invention the process is carried out in the presence of one or more suitable solvents. Suitable solvents will be specified below for the respective process steps.

Surprisingly, the pyrazoles of the formula (Ia) or (Ib) can be prepared under the inventive conditions only in several steps with good yields and in high purity, which means that the process according to the invention overcomes the abovementioned disadvantages of the preparation processes previously described in the prior art.

A further object of the present invention are intermediates of the general formula (IV)

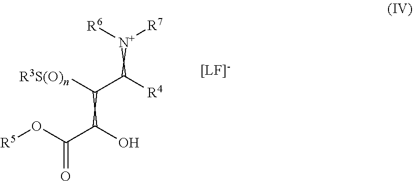

wherein

n, R³, R⁴, R⁵, R⁶ and R⁷ are defined as above and [LF]⁻ is an anion formed from a Lewis Acid [L] and one fluorine atom.

[LF]⁻ stands preferred for BF₄⁻, AlCl₃F⁻, SbCl₅F⁻, SbF₆⁻, PF₆⁻ or ZnCl₂F⁻, more preferred for BF₄, AlCl₃F⁻ or SbF₆⁻, even more preferred for BF₄⁻ or AlCl₃F⁻ and most preferred [LF]⁻ is BF₄.
Respectively the Lewis Acid [L] from which [LF]⁻ is formed, is preferably selected from BF₃, AlCl₃, SbCl₅, SbF₅, PF₅ or ZnCl₂ or any mixtures of those, more preferred from BF₃, AlCl₃ or SbF₅, even more preferred from BF₃ or AlCl₃ and most preferred the Lewis Acid is BF₃.

An object of the present invention are also disubstituted 5(3)-pyrazole carboxylates of the formula (Ia) or (Ib),

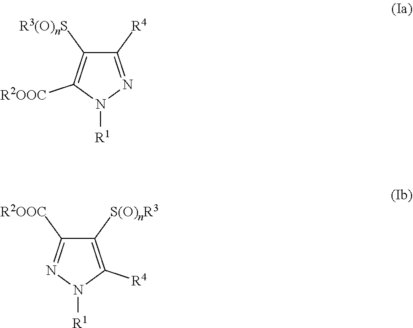

wherein
R¹ is selected from H, (C₁-C₆)alkyl, phenyl or 2-pyridyl,
R² is selected from (C₁-C₁₂)alkyl or (C₃-C₈)cycloalkyl,
R³ is selected from (C₁-C₁₂)alkyl, (C₁-C₃)haloalkyl, (C₃-C₈)cycloalkyl, (C₆-C₁₂)aryl, (C₁-C₃)alkyl(C₆-C₁₂)aryl and (C₆-C₁₂)aryl(C₁-C₆)alkyl,
R⁴ is selected from (C₁-C₆)haloalkyl and (C₁-C₃)haloalkoxy(C₁-C₆)haloalkyl and
n is 0, 1 or 2.

Preferred are disubstituted 5(3)-pyrazole carboxylates of the formula (Ia) or (Ib), wherein
R¹ is selected from H, (C₁-C₆)alkyl, phenyl or 2-pyridyl,
R² is selected from (C₁-C₆)alkyl or (C₃-C₆)cycloalkyl,
R³ is selected from (C₁-C₆)alkyl, (C₁-C₃)haloalkyl, (C₃-C₆)cycloalkyl, (C₆-C₉)aryl, (C₁-C₃)alkyl(C₆-C₉)aryl and (C₆-C₉)aryl(C₁-C₃)alkyl,
R⁴ is selected from (C₁-C₆)haloalkyl and (C₁-C₃)haloalkoxy(C₁-C₆)haloalkyl and
n is 0, 1 or 2.

More preferred are disubstituted 5(3)-pyrazole carboxylates of the formula (Ia) or (Ib), wherein
R¹ is selected from H, (C₁-C₆)alkyl or phenyl,
R² is selected from (C₁-C₆)alkyl,
R⁴ is selected from (C₁-C₆)haloalkyl and (C₁-C₃)haloalkoxy(C₁-C₆)haloalkyl, wherein the halogen is selected from fluoro and/or chloro and
n is 0, 1 or 2.

Even more preferred are disubstituted 5(3)-pyrazole carboxylates of the formula (Ia) or (Ib), wherein
R¹ is selected from H, methyl, ethyl or phenyl
R² is selected from methyl or ethyl,
R³ is selected from methyl or ethyl
R⁴ is selected from difluoromethyl (CF₂H), chlorofluoromethyl (CHFCl), 1,2,2,2-tetrafluoroethyl (CF₃CFH), pentafluoroethyl (C₂F₅) and trifluoromethoxyfluoromethyl (CF₃OCFH) and
n is 2.

Most preferred are disubstituted 5(3)-pyrazole carboxylates of the formula (Ia) or (Ib), wherein
R¹ is selected from H, methyl or phenyl,
R² is selected from methyl or ethyl,
R³ is methyl,
R⁴ is selected from difluoromethyl (CF₂H), chlorofluoromethyl (CHFCl), 1,2,2,2-tetrafluoroethyl (CF₃CFH), pentafluoroethyl (C₂F₅) and trifluoromethoxyfluoromethyl (CF₃OCFH) and
n is 2.

In a particularly preferred embodiment of the present invention n is 2 for the compounds of the general formula (Ia), (Ib) and (IV).

### General Definitions

In the context of the present invention, the term “halogen” (Hal), unless defined differently, comprises those elements which are selected from the group comprising fluorine, chlorine, bromine and iodine, preferably fluorine, chlorine and bromine, more preferably fluorine and chlorine.

Alkyl groups in the context of the present invention, unless defined differently, are linear or branched saturated hydrocarbyl groups. The definition C₁-C₁₂-alkyl encompasses the widest range defined herein for an alkyl group. Specifically, this definition encompasses, for example, the meanings of methyl, ethyl, n-, isopropyl, n-, iso-, sec- and t-butyl, n-pentyl, n-hexyl, 1,3-dimethylbutyl, 3,3-dimethylbutyl, n-heptyl, n-nonyl, n-decyl, n-undecyl or n-dodecyl.

The term Alkoxy, either on its own or else in combination with further terms, for example haloalkoxy, is understood in the present case to mean an O-alkyl radical, where the term “alkyl” is as defined above.

Cycloalkyl groups in the context of the present invention are monocyclic, saturated hydrocarbyl groups having 3 to 8 and preferably 3 to 6 carbon ring members, for example (but not limited to) cyclopropyl, cyclopentyl and cyclohexyl. This definition also applies to cycloalkyl as part of a composite substituent, for example cycloalkylalkyl etc., unless defined elsewhere.

Aryl groups in the context of the present invention, unless defined differently, are aromatic hydrocarbyl groups. The definition C₆₋₁₂-aryl encompasses the widest range defined herein for an aryl group having 6 to 12 skeleton atoms. The aryl groups may be mono- or bicyclic. Specifically, this definition encompasses, for example, the meanings of phenyl, cycloheptatrienyl, cyclooctatetraenyl, naphthyl and anthracenyl.

Arylalkyl groups (aralkyl groups) in the context of the present invention, unless defined differently, are alkyl groups which are substituted by aryl groups. Specifically, this definition encompasses, for example, the meanings of benzyl and phenylethyl.

Alkylaryl groups (alkaryl groups) in the context of the present invention, unless defined differently, are aryl groups which are substituted by one or more alkyl groups, which may have 1 to 6 carbon atoms in the alkyl chain. Specifically, this definition encompasses, for example, the meanings of tolyl or 2,3-, 2,4-, 2,5-, 2,6-, 3,4- or 3,5-dimethylphenyl.

Halogen-substituted radicals, for example haloalkyl, are mono- or polyhalogenated, up to the maximum number of possible substituents. In the case of polyhalogenation, the halogen atoms may be identical or different. Unless stated otherwise, optionally substituted radicals may be mono- or polysubstituted, where the substituents in the case of polysubstitutions may be the same or different.

Haloalkyl groups in the context of the present invention are straight-chain or branched alkyl groups having 1 to 6 and preferably 1 to 3 carbon atoms (as specified above), where some or all of the hydrogen atoms in these groups may be replaced by halogen atoms as specified above, for example (but not limited to) C₁-C₃-haloalkyl such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro, 2-difluoroethyl, 2,2-dichloro-2-fluoroethyl, 2,2,2-trichloroethyl, pentafluoroethyl and 1,1,1-trifluoroprop-2-yl. This definition also applies to haloalkyl as part of a composite substituent, for example haloalkylalkoxy, haloalkoxyhaloalkyl, haloalkylaminoalkyl etc., unless defined elsewhere. Preference is given to alkyl groups substituted by one or more halogen atoms, for example trifluoromethyl (CF₃), difluoromethyl (CHF₂), CF₃CFH, CF₃CH₂, CF₂C₁ CF₃CF₂, CF₃CCl₂.

The term intermediate used in the context of the present invention describes the substances which occur in the process according to the invention and are prepared for further chemical processing and are consumed or used therein in order to be converted to another substance. The intermediates can often be isolated and intermediately stored or are used without prior isolation in the subsequent reaction step. The term “intermediate” also encompasses the generally unstable and short-lived intermediates which occur transiently in multistage reactions (staged reactions) and to which local minima in the energy profile of the reaction can be assigned.

The inventive compounds may be present as mixtures of any different isomeric forms possible, especially of stereoisomers, for example E and Z isomers, threo and erythro isomers, and optical isomers, but if appropriate also of tautomers. Both the E and the Z isomers are disclosed and claimed, as are the threo and erythro isomers, and also the optical isomers, any mixtures of these isomers, and also the possible tautomeric forms.

Process Description

The process according to the invention is illustrated in Scheme 1:

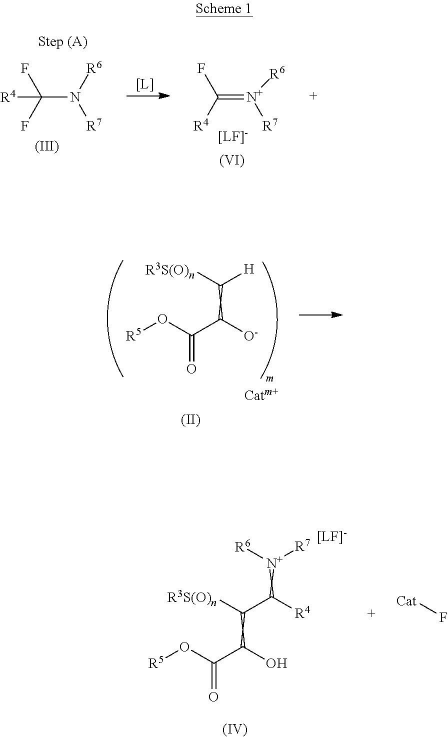

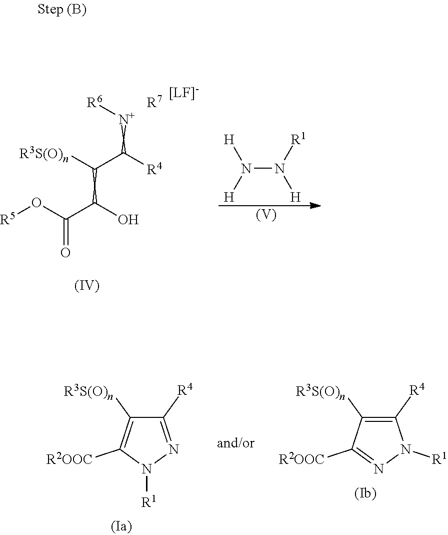

Step (A)

In step (A), FAR,s of the formula (III) are first converted into compounds of the formula (VI) in the presence of a Lewis Acid [L], and then reacted with compounds of the formula (II).

Preferred compounds of the general formula (III) are 1,1,2,2-tetrafluoroethyl-N,N-dimethylamine (TFEDMA), 1,1,2,2-tetrafluoroethyl-N,N-diethylamine, 1,1,2-trifluoro-2-(trifluoromethyl)ethyl-N,N-dimethylamine, 1,1,2-trifluoro-2-(trifluoromethyl)ethyl-N,N-diethylamine (Ishikawa's reagent), 1,1,2-trifluoro-2-chloroethyl-N,N-dimethylamine and 1,1,2-trifluoro-2-chloroethyl-N,N-diethylamine (Yarovenko's reagent), 1,1,2-trifluoro-N,N-dimethyl-2-(trifluoromethoxy)ethanamine. Compounds of the general formula (III) are used as iminoacylating reagents. Preference is given to 1,1,2,2-tetrafluoroethyl-N,N-dimethylamine (TFEDMA), 1,1,2-trifluoro-2-chloroethyl-N,N-dimethylamine and 1,1,2-trifluoro-N,N-dimethyl-2-(trifluoromethoxy)ethanamine.

α,α-Dihaloamines such as TFEDMA, Ishikawa's or Yarovenko reagents are commercially available or can be prepared according to (Yarovenko et al., Zh. Obshch. Khim. 1959, 29, 2159, Chem. Abstr. 1960, 54, 9724h or Petrov et al., J. Fluor. Chem. 109 (2011) 25-31). 1,1,2-trifluoro-N,N-dimethyl-2-(trifluoromethoxy)ethanamine is available according to S. Pazenok et al. Organic Letters (2017), 19(18), 4960-4963.

In the process according to the invention, the α,α-difluoroalkylamine (III) is first reacted with a Lewis acid [L] to form the compounds of formula (VI). The activation of α,α-difluoroalkylamines with Lewis acids is generally described in WO2008/022777.

Suitable Lewis Acids [L] according to the invention comprise all organic and inorganic, preferably inorganic, electronpair acceptors known by any person skilled in the art. Preferably the Lewis Acid is selected from BF₃, AlCl₃, SbCl₅, SbF₅, PF₅ or ZnCl₂ or any mixtures of those, more preferred from BF₃, AlCl₃ or SbF₅, even more preferred from BF₃ or AlCl₃ and most preferred the Lewis Acid is BF₃.

The Lewis acids can be used as a substance as such or as a stable solution in a suitable solvent, which is preferably the solvent used for step (A) in general.

Within the reaction of compounds of the formula (III) with the Lewis Acids to form compounds of formula (VI) an anion [LF]⁻ is formed from the Lewis Acid [L] and one fluorine atom of compound (III). [LF]⁻ stands preferred for BF₄⁻, AlCl₃F⁻, SbCl₅F⁻, SbF₆⁻, PF₆⁻ or ZnCl₂F⁻, more preferred for BF₄⁻, AlCl₃F⁻ or SbF₆⁻, even more preferred for BF₄⁻ or AlCl₃F⁻ and most preferred [LF]⁻ is BF₄.

According to the invention, 1 mol of the Lewis acid [L] is reacted with equimolar amounts of the α,α-difluoralkylamine of the formula (III).

The activated FAR (VI) is than reacted with compounds of formula (II) to obtain compounds of formula (IV).

In this step the compound of the formula (II) is preferably added to compound (VI) dissolved in a suitable solvent.

Compounds of formula (II) can be prepared from the cheap and available chemicals like methylalkylsulphones and oxalic acid esters according to Sokolov, M. P. et al; Journal of Organic Chemistry USSR (English Translation); vol. 22; (1986); p. 644-647. Preferred compounds of the formula (II) are sodium 3-methoxy-1-(methylsulfonyl)-3-oxoprop-1-en-2-olates, sodium 3-ethoxy-1-(methylsulfonyl)-3-oxoprop-1-en-2-olates, sodium 3-ethoxy-1-(phenylsulfonyl)-3-oxoprop-1-en-2-olates, potassium 3-methoxy-1-(methylsulfonyl)-3-oxoprop-1-en-2-olates, potassium 3-ethoxy-1-(methylsulfonyl)-3-oxoprop-1-en-2-olates.

For the process according to the invention 1 to 2 mol, preferred 1 to 1.5 mol, most preferred 1 to 1.2 mol of the activated FAR of the formula (VI) is reacted with 1 mol compound of formula (II).

According to the invention, the step (A) is preferably effected at temperatures of −20° C. to +60° C., more preferably at temperatures of −20° C. to +40° C., even more preferably at −10 to 20° C. and under standard pressure. Due to the hydrolytic sensitivity of the α,α-difluoroalkylamines, the reaction is preferably conducted in anhydrous conditions under inert gas atmosphere. The reaction time is not critical and may, according to the batch size and temperature, be selected within a range between a few minutes and several hours.

The reaction of compound (II) with the activated FAR (VI) is preferably effected in the presence of a base. Preference is given to organic bases, such as tri(C₁-C₄)alkylamines, pyridines, (C₁-C₄)alkylpyridines, for example picolines, and 1,8-diazabicyclo[5.4.0]undecene (DBU) or alkali metal hydroxides, for example lithium hydroxide, sodium hydroxide or potassium hydroxide, alkali metal carbonates, for example Na₂CO₃ or K₂CO₃ and alkali metal (C₁-C₄)alkoxides, for example NaOMe, NaOEt, NaOt-Bu or KOt-Bu, or alkali metal fluorides, for example KF. Also mixtures of those bases could be used. Most preferable are organic bases like pyridine and (C₁-C₄)alkypyridines or KF.

According to the invention preferably 1 to 5 mol, more preferably 1,5 to 4 mol and even more preferably 2 to 3.5 mol of the base are used for 1 mol of compound of the formula (II).

Step (A) is preferably carried out in the presence of one or more solvents. Suitable solvents for step (A) are, for example, aliphatic, alicyclic or aromatic hydrocarbons, for example petroleum ether, n-hexane, n-heptane, cyclohexane, methylcyclohexane, benzene, toluene, xylene or decalin, and halogenated hydrocarbons, for example chlorobenzene, dichlorobenzene, dichloromethane, chloroform, tetrachloromethane, dichloroethane or trichloroethane, ethers such as diethyl ether, diisopropyl ether, methyl tert-butyl ether, methyl tert-amyl ether, dioxane, tetrahydrofuran, 1,2-dimethoxyethane, 1,2-diethoxyethane or anisole, esters such as ethylacetate or isopropylacetat, nitriles such as acetonitrile, propionitrile, n- or isobutyronitrile or benzonitrile; amides such as N,N-dimethylformamide, N,N-dimethylacetamide, N-methylformanilide, N-methylpyrrolidone or hexamethylphosphoramide; sulphoxides such as dimethyl sulphoxide or sulphones such as sulpholane. Particular preference is given to THF, acetonitrile, methyl tert-butyl ether, dichloromethane, toluene, xylene, chlorobenzene, n-hexane, cyclohexane or methylcyclohexane, and very particular preference to acetonitrile, THF, methyl tert-butyl ether or dichloromethane.

The formed intermediates of the formula (W) can be used in the cyclization step (B) without prior workup. Alternatively, the intermediates can be isolated by suitable workup, characterized and optionally further purified.

Step (B):

In the cyclization step (B) compounds of formula (IV) are reacted with hydrazines of formula (V).

The reaction is preferably effected at temperatures of −20° C. to +80° C., more preferably at temperatures of +0° C. to +70° C., even more more preferably at +20 to +50° C. and under standard pressure. The reaction time is not critical and may, according to the batch size be selected within a relatively wide range.

According to the invention preferably 1 mol to 2 mol, more preferably 1 to 1.5 mol of the hydrazine are used for the conversion 1 mol of the compound of formula (IV).

Step (B) is preferably carried out in the presence of one or more solvents. More preferably the cyclization step (B) is effected without changing the solvent after step (A).

Suitable solvents are, for example, aliphatic, alicyclic or aromatic hydrocarbons, for example petroleum ether, n-hexane, n-heptane, cyclohexane, methylcyclohexane, benzene, toluene, xylene or decalin, and halogenated hydrocarbons, for example chlorobenzene, dichlorobenzene, dichloromethane, chloroform, tetrachloromethane, dichloroethane or trichloroethane, ethers such as diethyl ether, diisopropyl ether, methyl tert-butyl ether, methyl tert-amyl ether, dioxane, tetrahydrofuran, 1,2-dimethoxyethane, 1,2-diethoxyethane or anisole; alcohols such as methanol, ethanol, isopropanol or butanol, esters such as ethylacetate or isopropylacetat, nitriles such as acetonitrile, propionitrile, n- or isobutyronitrile or benzonitrile; amides such as N,N-dimethylformamide, N,N-dimethylacetamide, N-methylformanilide, N-methylpyrrolidone or hexamethylphosphoramide; sulphoxides such as dimethyl sulphoxide or sulphones such as sulpholane. Particular preference is given to acetonitrile, THF, methyl tert-butyl ether, ethanol, isopropylacetate, dichloromethane, toluene, xylene, chlorobenzene, n-hexane, cyclohexane or methylcyclohexane, and very particular preference to acetonitrile, THF, ethanol, isopropylacetate, toluene or xylene.

After the reaction has ended, the compounds of the general formula (I) can be isolated and purified by suitable methods known to any person skilled in the art. For example, the solvents could be removed and the product could be isolated by filtration. Alternatively the product could first be extracted with an organic solvent and washed with water, which is preferably acidified with an acid, preferably with HCl or H₂SO₄, then the solvent can be removed under reduced pressure and the product can be purified via crystallization.

Starting from compounds of the general formula (IV) two different isomers of the general formula (Ia) or (Ib) can be formed during step (B). The regioselectivity of the cyclization step (B) can be influenced by the choice of the solvents and substrates, especially of the hydrazine according to the general formula (V).

The compounds of the formula (I) where R²═(C₁-C₁₂)alkyl or (C₃-C₅)cycloalkyl can be converted in a further step (C) to pyrazole acids of the formula (I) with R²═H according to state of the art procedures as for example described in WO 2013/113829.

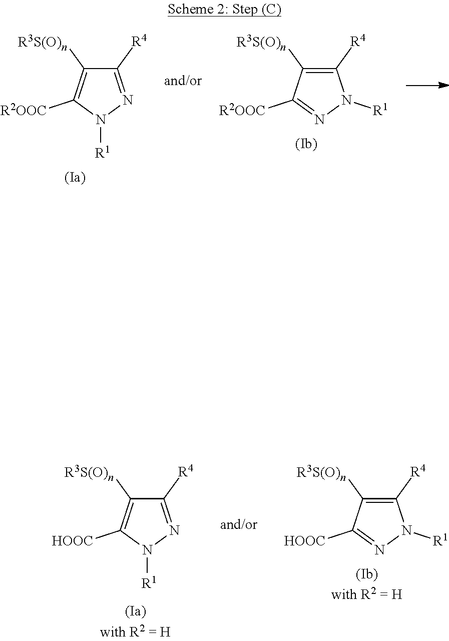

The process of the present invention preferably consists of steps A and B or steps A and B and C.

### EXAMPLES

The invention is illustrated by but not limited to the following examples:

### Example 1

### [(Z/E)-1-(difluoromethyl)-4-ethoxy-3-hydroxy-2-methyl-4-oxo-but-2-enylidene]-dimethyl-ammoniumtetrafluoroborate

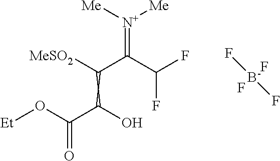

BF₃.OEt₂ (0.12 ml, 1.0 mmol) was added to a solution of 1,1,2,2-tetrafluoroethyl-N,N-dimethylamine (TFEDMA) (0.12 ml, 1.0 mmol) in dry dichloromethane (1 ml) under argon in a Teflon flask. The solution was stirred at room temperature for 15 min, before the dichloromethane was removed under reduced pressure. The solid residue was then taken up in acetonitrile (1 ml) and (0,216 g, 1.0 mmol) of sodium 3-ethoxy-1-(methylsulfonyl)-3-oxoprop-1-en-2-olate and (0,316 g, 4 mmol) pyridine was added in CD₃CN (2 ml) and the mixture was stirred at room temperature for 10 h. The solvent was evaporated in vacuum and the residue then analysed by ¹H, ¹³C and ¹⁹F NMR.

¹H NMR (600 MHz, CD₃CN-d₃) δ ppm 1.29 (br t, J=7.13 Hz, 4H) 3.22 (s, 3H) 3.44 (br s, 6H) 4.24 (q, J=7.23 Hz, 3H) 6.90-7.09 (m, 1H) 8.99-9.34 (m, 1H)

¹³C NMR (151 MHz, CD₃CN-d₃) δ ppm 14.20 (s, 1C) 46.42 (s, 1C) 46.65-47.52 (m, 1C) 62.96 (s, 1C) 104.90-105.03 (m, 1C) 111.25 (t, J=248.91 Hz, 1C) 165.03-165.52 (m, 1C) 166.44 (s, 1C) 177.33 (s, 1C).

¹⁹F NMR (CDCl₃, 282 MHz): δ ppm −117.3 (CHF₂, J_{F-H}=53.6 Hz), −150 (BF₄)

### Example 2

### Ethyl 5-(difluoromethyl)-2-methyl-4-methylsulfonyl-pyrazole-3-carboxylate

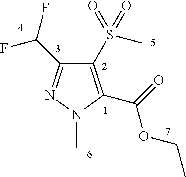 [SMILES: CCOC(=O)c1c(S(C)(=O)=O)c(C(F)F)nn1C]

BF₃CH₃CN complex (17 wt. % solution) (2.17 g, 20 mmol) was added to a solution of TFEDMA (2.86 g, 20 mmol) in 25 ml CH₃CN under argon in a Teflon flask. The solution was stirred at room temperature for 15 min, before (3.2 g, 15 mmol) of sodium 3-ethoxy-1-(methylsulfonyl)-3-oxoprop-1-en-2-olate and (4.7 g, 60 mmol) pyridine were added at 10° C. and the mixture was stirred at room temperature for 10 h. The mixture was cooled to −10° C. and (1.38 g 30 mmol) of methylhydrazine was added to the reaction mixture. The formed suspension was stirred for 4 h at RT and 100 ml of water were added. The product was extracted with ethylacetate, the organic solution washed with 10 ml (10 wt. %) HCl and water and the organic solvent was evaporated to furnish a pale yellow solid, which was recrystallized from methycyclohexane.

Yield 3.18 g, 75% of theory.

¹H NMR (DMSO-d₆, 600 MHz): δ ppm 7.25 (t, J=53.3 Hz, 1H) 4.43 (q, J=7.2 Hz, 2H) 4.09 (s, 3H) 1.36 (t, J=7.1 Hz, 3H)

¹³C NMR (151 MHz, DMSO-d₆) δ ppm 13.63 (s, 1C) 40.46 (s, 1C) 44.92 (s, 1C) 63.25 (s, 1C) 108.90 (t, J=236.56 Hz, 1C) 122.47 (s, 1C) 135.77 (s, 1C) 143.14 (t, J=24.85 Hz, 1C) 157.63 (s, 1C)

¹⁹F NMR (CFCl₃, 282 MHz): δ ppm −115.06, (d)

### Example 3

### 5-(difluoromethyl)-2-methyl-4-methylsulfonyl-pyrazole-3-carboxylic acid

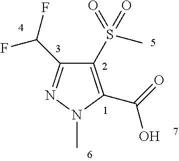 [SMILES: Cn1nc(C(F)F)c(S(C)(=O)=O)c1C(=O)O]

Ethyl 5-(difluoromethyl)-2-methyl-4-methylsulfonyl-pyrazole-3-carboxylate (2.83 g, 10 mmol) in toluene (15 ml) was mixed with an 8N aqueous sodium hydroxide solution (100 ml) and stirred at 50° C. for 3 h. The phases were separated and the water phase was acidified to pH 1 with 6N HCl. The formed precipitate was filtered off and dried.

Yield 2.3 g, 90%, colourless solid, m.p. 168° C.

¹H NMR (DMSO-d₆, 600 MHz): δ ppm 7.24 (t, J=53.3 Hz, 1H) 4.08 (s, 3H) 3.39 (s, 3H)

¹³C NMR (151 MHz, DMSO-d₆): δ ppm: 40.4 (s, 1C) 44.92 (s, 1C) 109.0 (t, J=236.56 Hz, 1C) 121.9 (s, 1C) 137.5 (s, 1C) 143.0 (t, J=24.85 Hz, 1C) 159.3 (s, 1C)

¹⁹F NMR (CFCl₃, 282 MHz): δ ppm −114.61, (d)

### Example 4

### Ethyl 5-(difluoromethyl)-1-methyl-4-methylsulfonyl-pyrazole-3-carboxylate

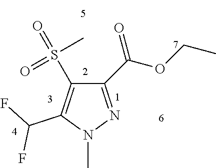 [SMILES: CCOC(=O)c1nn(C)c(C(F)F)c1S(C)(=O)=O]

BF₃CH₃CN complex (17 wt. % solution) (2.17 g, 20 mmol) was added to a solution of TFEDMA (2.86 g, 20 mmol) in 25 ml CH₃CN under argon in a Teflon flask. The solution was stirred at room temperature for 15 min, before the (3.2 g, 15 mmol) of sodium 3-ethoxy-1-(methylsulfonyl)-3-oxoprop-1-en-2-olate and (4.7 g, 60 mmol) of pyridine were added at 10° C. and the mixture was stirred at room temperature for 10 h. The solvent and volatile products were removed in vacuum (20 mbar) and the residue was taken up in 30 ml ethanol. The solution was cooled to 10° C. and (1.38 g, 30 mmol) of methylhydrazine was added to the reaction mixture within 30 min. The formed suspension was stirred for 4 h at RT and 100 ml of water were added. The product was extracted with ethylacetate, the organic solution was washed with 10 ml (10 wt. %) HCl and water and the organic solvent was evaporated to furnish a pale yellow solid, which was recrystallized from methycyclohexane.

Yield 3.3 g., 78% of theory.

¹H NMR (600 MHz, DMSO-d₆) δ ppm 1.32 (s, 3H) 3.50 (s, 3H) 4.11 (s, 3H) 4.34-4.38 (m, 2H) 7.42-7.61 (m, 1H).

¹³C NMR (151 MHz, DMSO-d₆) δ ppm 13.99 (s, 1C) 40.49 (s, 1C) 44.60 (s, 1C) 62.12 (s, 1C) 123.87 (s, 1C) 136.88 (s, 1C) 140.54-141.18 (m, 1C) 159.97 (s, 1C)

### Example 5

### Ethyl 5-(difluoromethyl)-1-methyl-4-methylsulfonyl-pyrazole-3-carboxylic acid

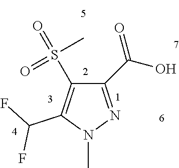 [SMILES: Cn1nc(C(=O)O)c(S(C)(=O)=O)c1C(F)F]

Ethyl 5-(difluoromethyl)-1-methyl-4-methylsulfonyl-pyrazole-3-carboxylate (2.83 g, 10 mmol) in toluene (15 ml) was mixed with an 8N aqueous sodium hydroxide solution (30 ml) and stirred at 50° C. for 3 h. The phases were separated and the water phase was acidified to pH 1 with 6N HCl. The formed precipitate was filtered off and dried.

Yield 2, 4 g, 95%, colourless solid, m.p. 186° C.

¹H NMR (600 MHz, DMSO-d₆) δ ppm 3.50 (s, 3H) 4.09 (s, 3H) 7.40-7.66 (m, 1H) 13.17-14.78 (m, 1H)

¹³C NMR (151 MHz, DMSO-d₆) δ ppm 40.46 (t, J=3.46 Hz, 1C) 44.50 (s, 1C) 107.21 (t, J=236.26 Hz, 1C) 123.66 (s, 1C) 136.68 (t, J=25.15 Hz, 1C) 142.02 (s, 1C) 161.55 (s, 1C)

¹⁹F NMR (CFCl₃, 282 MHz) δ ppm −115, 44, (d)

### Example 6

### Ethyl 5-(difluoromethyl)-4-methylsulfonyl-2-phenyl-pyrazole-3-carboxylate

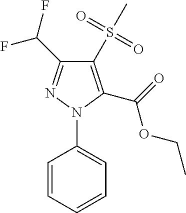 [SMILES: CCOC(=O)c1c(S(C)(=O)=O)c(C(F)F)nn1-c1ccccc1]

BF₃CH₃CN complex (17 wt. % solution) (3.26 g, 30 mmol) was added to a solution of TFEDMA (4.3 g, 30 mmol) in 25 ml CH₃CN under argon in a Teflon flask. The solution was stirred at room temperature for 15 min, before (3.2 g, 15 mmol) of sodium 3-ethoxy-1-(methylsulfonyl)-3-oxoprop-1-en-2-olate and (4.7 g, 60 mmol) pyridine were added at 10° C. and the mixture was stirred at room temperature for 10 h. The mixture was cooled to −10° C. and (3.2 g, 30 mmol) of Phenylhydrazine was added to the reaction mixture. The formed suspension was stirred for 4 h at room temperature and 100 ml of water were added. The product was extracted with ethylacetate, the organic solution washed with 10 ml (10 wt. %) HCl and water and the organic solvent was evaporated to furnish a pale yellow solid, which was recrystallized from ethanol/water.

Yield 4.17 g., 81% of theory.

1H NMR (600 MHz, DMSO-d₆) δ ppm 1.11 (t, J=7.13 Hz, 3H) 3.45 (s, 3H) 4.28 (q, J=7.16 Hz, 2H) 7.33-7.35 (m, 1H) 7.55-7.58 (m, 2H) 7.59-7.62 (m, 2H) 7.60-7.63 (m, 1H)

¹³C NMR (151 MHz, DMSO-d₆) δ ppm 13.66 (s, 1C) 40.49 (s, 1C) 44.95 (s, 1C) 63.28 (s, 1C) 108.92 (s, 1C) 122.48 (t, J=3.61 Hz, 1C) 135.79 (s, 1C) 142.97-143.38 (m, 1C) 157.65 (s, 1C)

### Example 7

### 5-(Difluoromethyl)-4-methylsulfonyl-2-phenyl-pyrazole-3-carboxylic acid

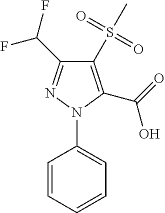 [SMILES: CS(=O)(=O)c1c(C(F)F)nn(-c2ccccc2)c1C(=O)O]

Ethyl 5-(difluoromethyl)-4-methylsulfonyl-2-phenyl-pyrazole-3-carboxylate (3.44 g, 10 mmol) in toluene (15 ml) was mixed with an 8N aqueous sodium hydroxide solution (142 ml) and stirred at 40° C. for 3 h. The phases were separated and water phase acidified to pH 1 with 6N HCl. The precipitate was filtered off and dried.

Yield 3 g, 95% of colourless solid, m.p. 187° C.

¹H NMR (600 MHz, DMSO-d₆) δ ppm 3.50 (s, 3H) 4.09 (s, 3H) 7.40-7.66 (m, 1H) 13.17-14.78 (m, 1H).

¹³C NMR (151 MHz, DMSO-d₆) δ ppm 40.46 (t, J=3.46 Hz, 1C) 44.50 (s, 1C) 107.21 (t, J=236.26 Hz, 1C) 123.66 (s, 1C) 136.68 (t, J=25.15 Hz, 1C) 142.02 (s, 1C) 161.55 (s, 1C)

1. Process for preparation of disubstituted 5(3)-pyrazole carboxylate of formula (Ia) or (Ib), 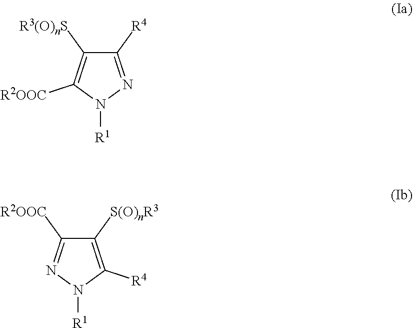
wherein
R¹ is selected from H, (C₁-C₆)alkyl, phenyl or 2-pyridyl,
R² is selected from H, (C₁-C₁₂)alkyl or (C₃-C₈)cycloalkyl,
R³ is selected from (C₁-C₁₂)alkyl, (C₁-C₃)haloalkyl, (C₃-C₈)cycloalkyl, (C₆-C₁₂)aryl, (C₁-C₃)alkyl(C₆-C₁₂)aryl and (C₆-C₁₂)aryl(C₁-C₆)alkyl,
R⁴ is selected from (C₁-C₆)haloalkyl and (C₁-C₃)haloalkoxy(C₁-C₆)haloalkyl and
n is 0, 1 or 2,
comprising (A), wherein an
α,α-difluoroalkylamino reagent (FAR) of formula (III), 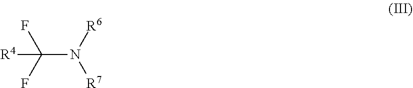
wherein
R⁴ is defined as above and
R⁶ and R⁷ are each independently selected from (C₁-C₆)alkyl, (C₃-C₈)cycloalkyl, (C₆-C₁₂)aryl or together with the nitrogen atom to which they are bonded may form a five- or six-membered ring,
is first converted into one or more compounds of formula (VI) in the presence of a Lewis Acid [L] 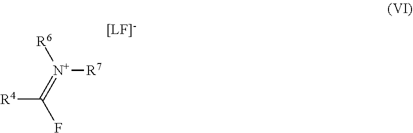
where R⁴, R⁶ and R⁷ are defined as for formula (III) and [LF]⁻ is an anion formed from the Lewis Acid [L] and one fluorine atom from compound (III),
and then the compound of formula (VI) is reacted with one or more enolates of formula (II), 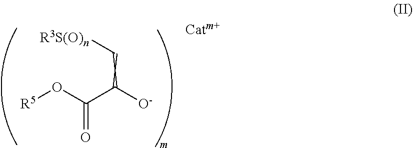
wherein
R⁵ is selected from (C₁-C₁₂)alkyl or (C₃-C₈)cycloalkyl,
R³ and n are defined as above,
m is 1 or 2 and
Cat^{m+} is selected from alkaline metal cations (with m=1), alkaline earth metal cations (with m=2), organic ammonium cations (with m=1) or organic phosphonium cations (with m=1),
to form the compound of formula (IV), 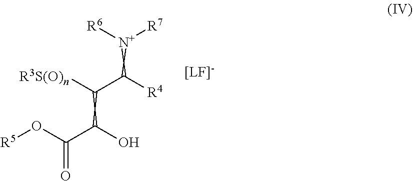
wherein
n, [LF]⁻, R³, R⁴, R⁵, R⁶ and R⁷ are defined as above,
and further comprising (B), wherein cyclization with a hydrazine of formula (V)
NH₂—NH—R¹ (V),
wherein R¹ is defined as above,
takes place to form the compound of formula (Ia) or (Ib).

2. Process according to claim 1, wherein the radicals in formula (Ia), (Ib), (II), (III), (IV), (V) and (VI) are defined as
R¹ is selected from H, (C₁-C₆)alkyl, phenyl or 2-pyridyl,
R² is selected from H, (C₁-C₆)alkyl or (C₃-C₆)cycloalkyl,
R³ is selected from (C₁-C₆)alkyl, (C₁-C₃)haloalkyl, (C₃-C₆)cycloalkyl, (C₆-C₉)aryl, (C₁-C₃)alkyl(C₆-C₉)aryl and (C₆-C₉)aryl(C₁-C₃)alkyl,
R⁴ is selected from (C₁-C₆)haloalkyl and (C₁-C₃)haloalkoxy(C₁-C₆)haloalkyl,
R⁵ is selected from (C₁-C₆)alkyl or (C₃-C₆)cycloalkyl,
n is 0, 1 or 2,
R⁶ and R⁷ are each independently selected from (C₁-C₆)alkyl, (C₃-C₆)cycloalkyl, (C₆-C₁₂)aryl or
R⁶ and R⁷ together with the nitrogen atom to which they are bonded may form a five- or six-membered ring,
m is 1 and
Cat^{m+} is selected from alkaline metal cations, optionally from Li⁺, Na⁺, K⁺ and Cs⁺, organic ammonium cations, optionally (R⁸)₄N⁺ or organic phosphonium cations, optionally (phenyl)₄P⁺, wherein
R⁸ are each independently selected from (C₁-C₆)alkyl.

3. Process according to claim 1, wherein the radicals in formula (Ia), (Ib), (II), (III), (IV), (V) and (VI) are defined as
R¹ is selected from H, (C₁-C₆)alkyl or phenyl,
R² is selected from H or (C₁-C₆)alkyl,
R³ is selected from (C₁-C₆)alkyl,
R⁴ is selected from (C₁-C₆)haloalkyl and (C₁-C₃)haloalkoxy(C₁-C₆)haloalkyl, wherein the halogen is selected from fluoro and/or chloro,
R⁵ is selected from (C₁-C₆)alkyl,
n is 0, 1 or 2,
R⁶ and R⁷ are each independently selected from (C₁-C₆)alkyl,
m is 1 and
Cat^{m+} is selected from Li⁺, Na⁺, K⁺, Cs⁺ or Me₄N⁺.

4. Process according to claim 1, wherein the radicals in formula (Ia), (Ib), (II), (III), (IV), (V) and (VI) are defined as
R¹ is selected from H, methyl, ethyl or phenyl
R² is selected from H, methyl or ethyl,
R³ is selected from methyl or ethyl
R⁴ is selected from difluoromethyl (CF₂H), chlorofluoromethyl (CHFCl), 1,2,2,2-tetrafluoroethyl (CF₃CFH), pentafluoroethyl (C₂F₅) and trifluoromethoxyfluoromethyl (CF₃OCFH),
R⁵ is selected from (C₁-C₃)alkyl,
n is 2,
R⁶ and R⁷ are methyl,
m is 1 and
Cat+ is selected from Li⁺, Na⁺ or K⁺.

5. Process according to claim 1, wherein the radicals in formula (Ia), (Ib), (II), (III), (IV), (V) and (VI) are defined as
R¹ is selected from H, methyl or phenyl,
R² is selected from H, methyl or ethyl,
R³ is methyl,
R⁴ is selected from difluoromethyl (CF₂H), chlorofluoromethyl (CHFCl), 1,2,2,2-tetrafluoroethyl (CF₃CFH), pentafluoroethyl (C₂F₅) and trifluoromethoxyfluoromethyl (CF₃OCFH),
R⁵ is selected from methyl or ethyl,
n is 2,
R⁶ and R⁷ are methyl,
m is 1 and
Cat^{m+} is Na⁺ or K⁺.

6. The process according to claim 1, wherein the compound of formula (III) is selected from 1,1,2,2-tetrafluoroethyl-N, N-dimethylamine (TFEDMA), 1,1,2,2-tetrafluoroethyl-N, N-diethylamine, 1,1,2-trifluoro-2-(trifluoromethyl)ethyl-N, N-dimethylamine, 1,1,2-trifluoro-2-(trifluoromethyl)ethyl-N, N-diethylamine (Ishikawa's reagent), 1,1,2-trifluoro-2-chloroethyl-N,N-dimethylamine and 1,1,2-trifluoro-2-chloroethyl-N,N-diethylamine (Yarovenko's reagent) or 1,1,2-trifluoro-N, N-dimethyl-2-(trifluoromethoxy)ethanamine.

7. The process according to claim 1, wherein the Lewis Acid is selected from BF₃, AlCl₃, SbCl₅, SbF₅, PF₅ or ZnCl₂.

8. The process according to claim 1, wherein [LF]⁻ stands for BF₄, AlCl₃F⁻, SbCl₅F⁻, SbF₆⁻, PF₆⁻ or ZnCl₂F⁻.

9. The process according to claim 1, wherein (A) is effected at one or more temperatures of −20° C. to +60° C.

10. The process according to claim 1, wherein reaction of compound (II) with the activated FAR (VI) is optionally effected in presence of a base.

11. The process according to claim 10, wherein the base is selected from tri(C₁-C₄)alkylamines, pyridines, (C₁-C₄)alkylpyridines, 8-diazabicyclo[5.4.0]undecene (DBU), alkali metal hydroxides, alkali metal carbonates, alkali metal (C₁-C₄)alkoxides or alkali metal fluorides.

12. The process according to claim 1, wherein (B) is effected at one or more temperatures of −20° C. to +80° C.

13. The process according to claim 1, wherein the process is carried out in the presence of a suitable solvent and (B) is effected without changing solvent after (A).

14. Intermediate of formula (IV) 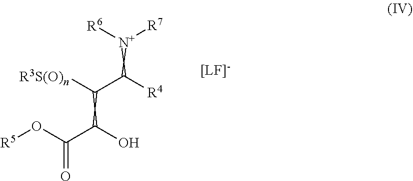
Wherein
R¹ is selected from H, (C₁-C₆)alkyl, phenyl or 2-pyridyl,
R² is selected from H, (C₁-C₁₂)alkyl or (C₃-C₈)cycloalkyl,
R³ is selected from (C₁-C₁₂)alkyl, (C₁-C₃)haloalkyl, (C₃-C₈)cycloalkyl, (C₆-C₁₂)aryl, (C₁-C₃)alkyl(C₆-C₁₂)aryl and (C₆-C₁₂)aryl(C₁-C₆)alkyl,
R⁴ is selected from (C₁-C₆)haloalkyl and (C₁-C₃)haloalkoxy(C₁-C₆)haloalkyl and
n is 0, 1 or 2,
R⁶ and R⁷ are each independently selected from (C₁-C₆)alkyl, (C₃-C₈)cycloalkyl, (C₆-C₁₂)aryl or together with the nitrogen atom to which they are bonded may form a five- or six-membered ring,
and [LF]⁻ is an anion formed from a Lewis Acid [L] and one fluorine atom.

15. Intermediate according to claim 14, wherein [LF]⁻ stands for BF₄⁻.

16. Disubstituted 5(3)-pyrazole carboxylate of formula (Ia) or (Ib), 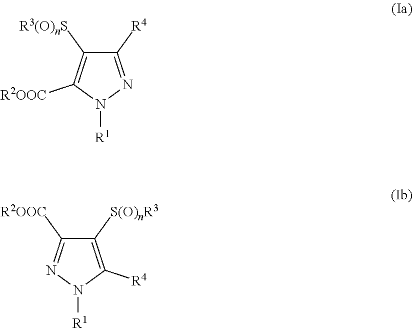
wherein
R¹ is selected from H, (C₁-C₆)alkyl, phenyl or 2-pyridyl,
R² is selected from (C₁-C₁₂)alkyl or (C₃-C₈)cycloalkyl,
R³ is selected from (C₁-C₁₂)alkyl, (C₁-C₃)haloalkyl, (C₃-C₈)cycloalkyl, (C₆-C₁₂)aryl, (C₁-C₃)alkyl(C₆-C₁₂)aryl and (C₆-C₁₂)aryl(C₁-C₆)alkyl,
R⁴ is selected from (C₁-C₆)haloalkyl and (C₁-C₃)haloalkoxy(C₁-C₆)haloalkyl and
n is 0, 1 or 2.

17. Disubstituted 5(3)-pyrazole carboxylate of formula (Ia) or (Ib) according to claim 16, wherein
R¹ is selected from H, (C₁-C₆)alkyl, phenyl or 2-pyridyl,
R² is selected from (C₁-C₆)alkyl or (C₃-C₆)cycloalkyl,
R³ is selected from (C₁-C₆)alkyl, (C₁-C₃)haloalkyl, (C₃-C₆)cycloalkyl, (C₆-C₉)aryl, (C₁-C₃)alkyl(C₆-C₉)aryl and (C₆-C₉)aryl(C₁-C₃)alkyl,
R⁴ is selected from (C₁-C₆)haloalkyl and (C₁-C₃)haloalkoxy(C₁-C₆)haloalkyl and
n is 0, 1 or 2.

18. Disubstituted 5(3)-pyrazole carboxylate of formula (Ia) or (Ib) according to claim 16, wherein
R¹ is selected from H, (C₁-C₆)alkyl or phenyl,
R² is selected from (C₁-C₆)alkyl,
R³ is selected from (C₁-C₆)alkyl,
R⁴ is selected from (C₁-C₆)haloalkyl and (C₁-C₃)haloalkoxy(C₁-C₆)haloalkyl, wherein the halogen is selected from fluoro and/or chloro and
n is 0, 1 or 2.

19. Disubstituted 5(3)-pyrazole carboxylate of formula (Ia) or (Ib) according to claim 16, wherein
R¹ is selected from H, methyl, ethyl or phenyl
R² is selected from methyl or ethyl,
R³ is selected from methyl or ethyl,
R⁴ is selected from difluoromethyl (CF₂H), chlorofluoromethyl (CHFCl), 1,2,2,2-tetrafluoroethyl (CF₃CFH), pentafluoroethyl (C₂F₅) and trifluoromethoxyfluoromethyl (CF₃OCFH) and
n is 2.

20. Disubstituted 5(3)-pyrazole carboxylate of formula (Ia) or (Ib) according to claim 16, wherein
R¹ is selected from H, methyl or phenyl,
R² is selected from methyl or ethyl,
R³ is methyl,
R⁴ is selected from difluoromethyl (CF₂H), chlorofluoromethyl (CHFCl), 1,2,2,2-tetrafluoroethyl (CF₃CFH), pentafluoroethyl (C₂F₅) and trifluoromethoxyfluoromethyl (CF₃OCFH) and
n is 2.
